(19) United States
(12) Patent Application Publication
(10) Pub. No.: US 2007/0260136 A1
(43) Pub. Date: Nov. 8, 2007

(54) Method For Conducting Double-Blind Testing Using A Constant Amplitude Electromagnetic System

(75) Inventors: Clifford Wayne Hunter, Houston, TX (US); Robert Emmett Ward JR., Houston, TX (US); Donald Coolidge Wunsch, Albuquerque, NM (US)

Correspondence Address: BUSKOP LAW GROUP, P.C., 4511 Dacoma Street, HOUSTON, TX, 77092, US

(73) Assignee: KSM, Inc.

(21) Appl. No.: 11/558,879

(22) Filed: Nov. 10, 2006

Related U.S. Application Data
(63) Continuation-in-part of application No. 11/408,408, filed on Apr. 21, 2006.
(60) Provisional application No. 60/673,398, filed on Apr. 21, 2005.

(51) Int. Cl. A61B 5/05

(52) U.S. Cl. 600/407

(57) ABSTRACT

A method for performing a randomized double-blind medical clinical trial on a statistically significant number of participants using a constant amplitude electromagnetic system. The constant amplitude electromagnetic system comprises: a housing; at least one magnetic module contained within the housing, wherein each magnetic module includes: a ferromagnetic housing; including a non ferromagnetic segment forming an aperture; a wound magnetic wire coil on a perforated spindle having a coil diameter greater than a coil depth surrounding the aperture and located between the non-ferromagnetic segment and the ferromagnetic housing. The wound magnetic wire coil generates a constant amplitude electromagnetic magnetic field within the aperture when coupled to a direct electrical current.

### CROSS-REFERENCE TO RELATED APPLICATIONS

This patent application is a continuation-in-part application that claims the benefit, under 35 USC §120, of the co-pending prior non-provisional application Ser. No. 11/408,408, which was filled Apr. 21, 2006; which claimed priority to a co-pending provisional application 60/673,398, which was filed on Apr. 21, 2005. The prior co-pending non-provisional application and prior provisional application are incorporated by reference along with their appendices.

### FIELD

The present embodiments relate generally to a method for conducting a double-blind test using a constant amplitude electromagnetic system.

### BACKGROUND

The invention will be described with reference to the drawings, the detailed description, and the appended claims.

There exists a need for a method for conducting a double-blind test using a constant amplitude electromagnetic system. The method for conducting the double-blind test must fulfill the federal regulatory standards and be accepted by the medical community.

There exists a need for a double-blind test using the constant amplitude electromagnetic system that produces results in participants in a relatively short time.

There exists a need for a method for conducting a double-blind that is capable of treating a participant in a consistent repeatable manner regardless of the training of the operator.

There exists a need for a method for conducting a double-blind test utilizing the constant amplitude electromagnetic system that maintains a substantially similar temperature in each magnetic module. It is important to maintain a substantially similar temperature within each of the modules so that the participant is not alerted to a temperature difference which would indicate that the magnetic module was on or off.

The present embodiments meet these needs.

### BRIEF DESCRIPTION OF THE DRAWINGS

The detailed description will be better understood in conjunction with the accompanying drawings as follows:

FIG. 1 is a left and right view of an embodiment of the housing with out the magnetic modules.

FIG. 2 depicts an embodiment of a left and right view of the housing with the magnetic modules.

FIG. 3 depicts a left and right view of the inside of the housing.

FIG. 4 is a depiction of an embodiment of a magnetic module.

FIG. 5 is a schematic of an embodiment of the continuous flow cooling system.

FIG. 6 is an electronic schematic of an embodiment of the electric system.

FIG. 7 is a depiction of an embodiment of the perforated spindle.

FIG. 8 illustrates a schematic of an embodiment of an electrical connector.

The present embodiments are detailed below with reference to the listed Figures.

### DETAILED DESCRIPTION OF THE EMBODIMENTS

Before explaining the present embodiments in detail, it is to be understood that the embodiments are not limited to the particular embodiments and that they can be practiced or carried out in various ways.

The invention relates to a method for performing a randomized double-blind medical clinical trial on a statistically significant number of participants using a constant amplitude electromagnetic system. Particularly, the constant amplitude electromagnetic treatment system provides a constant amplitude electromagnetic field.

An embodiment of the method for performing a randomized double-blind medical clinical trial on a significant number of participants is used to determine how the constant amplitude electromagnetic field will affect defined medical ailments. The medical ailments can be arthritis, diabetes, HIV, AIDS, shingles, soft tissue damage, sickle cell anemia, cardiopulmonary disease, cancer, chronic pain, or a similar disease.

An embodiment of the method for performing a medical clinical trial on a statistically significant number of participants using a constant amplitude electromagnetic system includes using a constant amplitude electromagnetic system, which is described herein.

The embodiment of the method can further include forming a medical protocol for use with the constant amplitude electromagnetic system to determine if exposure to a constant amplitude electromagnetic field will affect a defined ailment.

The medical protocol should be followed to select the required participant criteria for receiving an actual treatment using the constant amplitude electromagnetic system.

Depending on the medical ailment to be tested, a participant must meet certain criteria. If the participant does not meet the criteria he will not be eligible to participate in the randomized double-blind medical clinical trial.

Once the participant is selected, a random number generator is used. The random number generator classifies the participant. This classification determines if the participant receives treatment using the medical protocol or receives a placebo treatment.

When a statistically significant number of participants are determined to meet the participant criteria, each of the participants will be assigned a participant number.

The method further includes using a random number generator to classify which of the participants receives the actual treatment, and which receives a placebo treatment.

Each of the statistically significant number of participants will have a treatment provided to them, which is based on the random number generator classification. This treatment can include actual treatment using the constant amplitude electromagnetic system, or placebo treatment using the constant amplitude electromagnetic system where the magnetic module is not coupled to a direct current.

If the participant is being treated using the medical protocol, the participant will be exposed to a constant amplitude electromagnetic field. The constant amplitude electromagnetic field is provided using the constant amplitude electromagnetic system.

A participant receiving actual treatment can be treated as described herein.

If the participant is receiving placebo treatment, the participant will be treated in the same manner as a participant receiving actual treatment, except the participant receiving placebo treatment will not be exposed to the constant amplitude electromagnetic field.

The significant number of participants will not be advised of the treatment they are receiving. They will not know if they are receiving the actual treatment or the placebo treatment.

Once the tests are performed, data will be analyzed using a technician without knowledge of which of the statistical significant number of participants received actual treatment or placebo treatment.

The analyzed data will be stored and correlated using the participant numbers.

It is advisable that during the randomized double-blind medical clinical trial that the constant amplitude electromagnetic system not be modifiable by the operator of the constant amplitude electromagnetic field system.

The data that the technician can examine can include blood samples, taken before and after the treatment; pain scale measurements; interview with the participant regarding whether the pain or numbness has subsided, or other similar objective or subjective methods of evaluating the participants condition.

The Provisional Application, incorporated by reference in its entirety, provides additional examples in the form of photographs and schematics related to the present invention.

The actual treatment selectively includes exposing the end of a limb of a participant to the constant amplitude electromagnetic field for a predetermined length of time. For the actual treatment to be effective the predetermined length of time can range from approximately 10 minutes to 3 hours.

It is contemplated that the actual treatment could provide from 2 to 4 constant amplitude electromagnetic fields, with each of the constant amplitude electromagnetic fields being adapted to treat the end of the limb of a participant.

The constant amplitude electromagnetic field can have an intensity from 50 to 1000 gauss at a center location where treatment occurs. The intensity of the constant amplitude electromagnetic field declines in intensity as the distance from the center location increases. The electromagnetic field can decline from 80% to 90% of its original intensity at a location approximately 2 feet from the center location.

The constant amplitude electromagnetic field has constant directional vector electromagnetic gradients inward and outward from the treatment volume. The constant amplitude electromagnetic field is concentrated at the center location.

During the exposure, the constant amplitude electromagnetic field simultaneously treats venous and arterial blood cells as well as nerve endings in the limb of the participant. Once the actual treatment is provided for a length of time ranging from 10 minutes to about 3 hours, the exposure of the end of the limb of the participant is stopped. The exposure is stopped by removing the end of the limb from the center location.

The actual treatment can include using a continuous flow cooling system, which continuously maintains a temperature at lees than 105 degrees Fahrenheit at an area around the constant amplitude electromagnetic field. The temperature can be continuously maintained using a cooling fluid.

The continuous flow cooling system can further include periodically removing heat from the cooling fluid to the external environment.

In an alternative embodiment, the cooling fluid can be a hydrotreated light naphthenic petroleum oil. The cooling fluid, in the continuous flow cooling system is used to maintain the temperature within the magnetic module at an appropriate level. The appropriate level can be from the ambient temperature to less than 105 degrees Fahrenheit. The ambient temperature should be from 68 degrees Fahrenheit to 80 degrees Fahrenheit.

In another alternative, non-limiting embodiment of the actual treatment, the constant amplitude electromagnetic field can be automatically stopped when the temperature in the area around the constant amplitude electromagnetic field exceeds a pre-set limit.

It is additionally contemplated that when multiple constant amplitude electromagnetic fields are used, one field can be off while others are in use.

It is contemplated that each constant amplitude electromagnetic field can be adapted to treat limbs of different participants simultaneously.

In an alternative embodiment, it is contemplated that the participant can be a human or an animal.

In addition to a method for treating a participant, the invention relates to a constant amplitude electromagnetic system for treating an end of a limb of a participant. A non-limiting embodiment of the constant amplitude electromagnetic treatment system typically contains four wound magnetic wire coils each with an opening (or aperture) at its center that allows a body part or appendage, i.e., a hand, arm, foot, or leg, to be suitably positioned to efficiently be exposed to a continuous DC constant amplitude electromagnetic field.

Features of a non-limiting constant amplitude electromagnetic treatment system include, but are not limited to safety locked controls; an integrated continuous flow cooling system; the use of static electromagnetic fields, which produce minimal extraneous electromagnetic fields; non-invasive participant treatment; simple non-adjustable on-off controls; operation status metering for each magnetic module; operation of standard 220 volt wall power, electrical safety tested, and wheel mounted or in the alternative the wall power can be can be 230 volt AC, 20 amperes, 50 Hz.

The Provisional Application, incorporated by reference in its entirety, provides additional examples in the form of photographs and schematics related to the present invention.

In certain embodiments of the invention described below and in the figures, a system may be approximately 6½′×2½′×4′ high and weigh in excess of 2000 pounds. The system may contain four wound magnetic wire coils with central openings of 4.25″, 4.25″, 6.25″, and 8.25″ diameters and in certain embodiments approximately 4″ in length. The system is powered by single phase, 220 volt AC or can be powered using 230 volt AC, 20 amperes, 50 Hz. The AC is rectified to approximately 220 volt DC by separate full-wave bridge rectifier circuitry which is connected to each wound magnetic wire coil. AC power is supplied to the pump and fan of the switchable continuous cooling system. The system is connected by a 220-volt, 20 ampere, three wire, grounded, UL approved power cable. In operation each wound magnetic wire coil draws approximately 2.3 amperes, and the system with all systems operating draws approximately 17 amperes. AC power switches are separately provided for main power, each wound magnetic wire coil, the pump, and the fan. The wound magnetic wire coils may be surrounded, with the exception of the aperture, by a closed ferromagnetic housing. Other alternative materials can be used but are not as good. Such materials could include stainless steel, aluminum, or titanium. The ferromagnetic housing may be filled with dielectric oil for additional insulation and cooling. All wound magnetic wire coils, the pump, the fan, and the power circuitry housings are solidly electrically grounded to the metal structural housing.

The housing is electrically connected to the ground wire of the power cable. All operating features are typically completely controlled by on-off switches. Computer microprocessor or software systems need not be employed. The main electrical function is to provide DC Current to the wound magnetic wire coils. All circuitry is of standard design and implemented with proven robust conservatively rated components.

The constant amplitude electromagnetic system can have a lockable front cover. There are several contemplated controls and meters for operating the constant amplitude electromagnetic field system.

All controls can be located behind a hinged, lockable front panel. During operation it is contemplated that the panel remain unlocked for quick access to all controls.

The system controls are typically simple on-off switches, however it is contemplated that variable types of switches can be used. There is no apparent need for variable or adjustable controls. There may be various types of switches including one or more switches separately controlling the power to a wound magnetic wire coil. Each switch should be clearly labeled to indicate the magnetic module it controls, at least one second switch controls the switchable continuous flow cooling system, and a third switch may control the cooling fan for the heat exchanger. It is advisable that when a magnetic module is on, both the fan and pump are also on.

Adjacent to each switch is an ammeter that indicates the current being drawn to the load controlled by that switch. Each ammeter is also separately labeled with the same number as to its related switch and load. The meters are physically located so that they cannot be easily confused. The meters for the switches to the left are located to the left of the corresponding switches and the meters for the switches to the right are located to the right of the corresponding switches. The meters are all identical to avoid confusion. They read to a maximum of 10 amperes of AC current with subdivisions of 2 amperes. The four meters that indicate the power to the magnetic coils measure the AC currents being supplied before rectification. The pump and fan ammeters measure the AC current drawn by the respective units. A temperature monitoring system, such as a thermometer, may also be installed in or around an aperture or the magnetic wire coil to measure the temperature at each magnetic module.

The system may be powered by 220 volt AC, 20 amperes, 60 Hz. In another, non-limiting alternative embodiment, the system can be powered using 230 volt AC, 20 amperes, 50 Hz. Main power switches may include, but are not limited to individual on-off switch for each operating magnetic module, the pump, and the fan. There may also be provided a locked key access to the control panel or some other mechanism to regulate access to the controls.

The system may further comprise dielectric insulation including insulation with wide safety margins, particularly between active coils and the participant. The insulation may include wiring dielectric interfaces and dielectric insulation oil. The oil may also serve as a coolant. A participant's arm or leg may rest within a dielectric cuff.

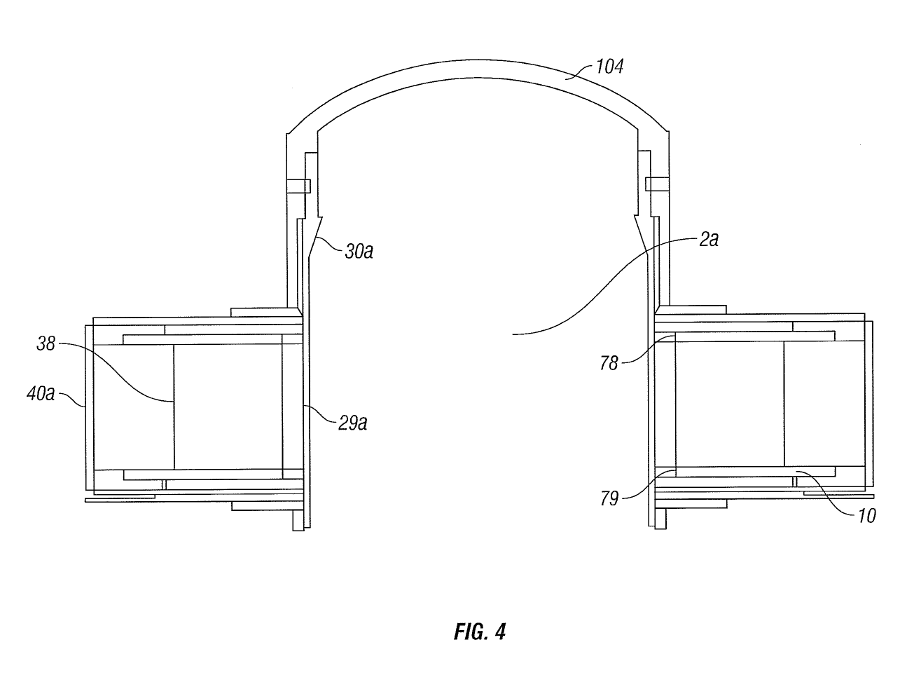

Typically, AC current will be rectified to DC current before powering the magnetic modules. The AC power is typically connected to full-wave bridge rectifier circuit(s) that provide the DC voltage to the magnetic coil(s) through control switches. An exemplary electrical schematic is illustrated in FIG. 4.

The constant amplitude electromagnetic system exposes an end of a limb of a participant selectively disposed within an aperture to a DC constant amplitude electromagnetic field. For safety considerations the magnitude of this field can be compared to the magnetic fields approved for use in Magnetic Resonance Imaging (MRI) and Magnetic Resonance Spectroscopy (MRS) diagnostic systems. The magnetic fields in such systems can expose the whole participant body to levels up to 100,000 gauss. The constant amplitude electromagnetic system exposes only extremities of the participant to levels significantly below those of a typical MRI system. The wound magnetic wire coil, being a solenoid and being powered by direct current, produces an electromagnetic field which rapidly decreases in intensity within short distances from the end of the wound magnetic wire coil.

The constant amplitude electromagnetic system can include several mechanical features. The switchable continuous flow cooling system uses a pump to force cooling fluid into each of the magnetic modules. A non-limiting example of a coolant circulation pump is a Model TE-6MD-SC-c-csa 586504 (Little Giant Pump Company). The Pump can be powered by a ½ hp motor with electrical requirements of 230 volts, 60 Hz, 4.5 amps. All electrical power is provided with hard wire direct connections internal to the constant amplitude electromagnetic system. The power to the pump may be controlled by a control switch on the system control panel.

The switchable continuous flow cooling system also uses a fan as part of the heat exchanger. An exemplary cooling fan is manufactured by Thermal Transfer Products LTD. The power to the fan may be controlled by a control switch on the system control panel.

An exemplary heat exchanger is manufactured by thermal Transfer Products LTD. Typical elements and coolant paths are illustrated by the schematic of the switchable continuous cooling system in FIG. 3.

The constant amplitude electromagnetic system may be operated in the following way. The system's power cable may be connected to a standard GFI wall outlet rated at 220-volt and 30 amperes. It is advisable that the outlet be fed from a 220-volt, single phase, minimum 20-amperes source fused or over current breaker rated for 20 amperes. The room air conditioning system should be capable of maintaining an ambient temperature from 68 degrees Fahrenheit to 80 degrees Fahrenheit which optimizes the capabilities of the switchable continuous flow cooling system and provides for the participant's comfort.

Once the system is plugged into an appropriate electrical outlet. The operator should first switch on the switchable continuous flow cooling system, then the operator should turn on the magnetic modules separately. The operator should monitor temperature gauges and check that the temperature does not exceed prescribed limits, such as 10 percent of the ambient temperature.

The switchable continuous flow cooling system should be engaged prior to the system being operated for any substantial length of time. The switchable continuous flow cooling system is designed to provide for removal of heat generated when the wound magnetic wire coils generate the constant amplitude electromagnetic field.

The non-flowing cooling fluid within each ferromagnetic housing will cool the wound magnetic wire coils for a substantial length of time. However, an operator should not allow the magnetic modules to be turned on for a significant period of time without turning on the switchable continuous flow cooling system.

The operator, when operating the constant amplitude electromagnetic system, should ensure the safety of the constant amplitude electromagnetic system at all times.

Exemplary safety measures include electrical safety tests using a QuadTech, Model Guardian 6100, Medical Production Safety Analyzer with current calibration certification. Safety test measurements that can be made include to (1) Ground Continuity, (2) Ground Bonding, (3) AC Hipot, (4) DC Hipot, (5) AC insulation resistance, (6) DC Insulation Resistance, and (7) Line Leakage Current.

In addition, the operator should always ensure that constant amplitude electromagnetic treatment system is maintained to operate as designed. This means the operator should ensure that the constant amplitude electromagnetic treatment system should always meet certain standards including the following: International Medical System Directives, ISO 60601-1, ISO 60601-1-1, 60601-1-2; medical functional and safety standards, ISO 14971; system risk analysis; and ISO 9000/ISO 13485 Quality Assurance Standards. These standards as referenced are the current 2006 standards. This ensures that each constant amplitude electromagnetic treatment system operates substantially similar and provides identical treatment conditions.

In the event that the constant amplitude electromagnetic system of the invention has not been used for an extended period of time it should be prepared and stored in an appropriate manner.

This includes at a minimum: (a) ensure the control panel is key locked and that the key is not left accessible with the system, (b) ensure that the power cable is coiled and attached to the system in a manner such that it will not come free, drag on the floor, or drag within the wheel area of the system, (c) make sure the cable and connector cannot be bent or crushed as a result of the placement of the system for storage, (d) cover the system completely in a maimer to prevent the dust or water from reasonably coming into contact with the system, and (e) store the system in a temperature controlled environment or in an area in which the temperature remains in the range of 40 degrees Fahrenheit to 120 degrees Fahrenheit.

In another alternative embodiment of the constant amplitude electromagnetic system, the constant amplitude electromagnetic system has a ferromagnetic housing having at least one magnetic module. Each of the magnetic modules can have an aperture, which is sized to receive at least one limb.

The constant amplitude electromagnetic system can further include a non-ferromagnetic segment forming the apertures. Furthermore, a tubular sleeve is disposed within an aperture. The tubular sleeve can have a diameter ranging from 3 to 10 inches. It is further contemplated that the tubular sleeve can have an end cap.

The magnetic modules include a wound magnetic wire coil operatively connected to a perforated spindle. The wound magnetic wire coil has a coil diameter, which is greater than the magnetic wire coil's coil depth. The magnetic wire coil can surround each aperture.

It is also contemplated that the magnetic wire coil can be contained between the non-ferromagnetic segment and the ferromagnetic housing.

The wound magnetic wire coil generates a constant amplitude electromagnetic field within the aperture when coupled to a direct electrical current. The constant amplitude electromagnetic field is concentrated in the center of the aperture.

In addition, the system includes a switchable continuous flow cooling system. It is contemplated that the switchable continuous flow coiling system simultaneously cools each wound magnetic wire coil within the ferromagnetic housing.

The switchable continuous flow cooling system can have a first cooling fluid reservoir. The first cooling fluid reservoir can be in fluid communication with a pump. The cooling fluid flows from the first cooling fluid reservoir to a pressurizable cooling fluid reservoir. The cooling fluid can then flow from the pressurizable cooling fluid reservoir to each wound magnetic wire coil.

The cooling fluid flows around each of the wound magnetic wire coils, cooling the wound magnetic wire coils. The cooling fluid is used to maintain a substantially similar temperature in each of the magnetic modules; the temperature can range from ambient temperature to 105 degrees Fahrenheit. The cooling fluid will flow from the ferromagnetic housing to a heat exchanger. The heat exchanger can transfer heat from the cooling fluid to the outside environment. It is additionally contemplated that the heat exchanger can be a fin, fan heat exchanger. After passing through the heat exchanger, the fluid can return to the first cooling fluid reservoir.

Each perforated spindle of the wound magnetic wire coils can have a plurality of elliptical openings. The openings allow the cooling fluid to surround the wound magnetic wire coil, thereby providing continuous convection cooling. It is contemplated that the cooling fluid can be a dielectric cooling fluid, such as a hydrotreated light naphthenic petroleum oil.

A plurality of spacers separate the perforated spindle from the non-ferromagnetic segment.

It is contemplated that the pump, which pressurizes the cooling fluid in the pressurizable reservoir, can be a switchable magnetic coupling fluid circulation pump. It is contemplated that the pump will not generate more than 20 psi of pressure.

The system can further include a plurality of switches to individually couple electrical current to each magnetic module.

It is contemplated that the tubular sleeve can be formed of an insulating material. The insulating material can be PVC, a non-toxic hard crystalline polymer, polyethylene, polypropylene, polyamide, a homopolymer of one of these polymers, a copolymer of one of these polymers, or combinations thereof.

The constant amplitude electromagnetic field is generally a static field but can contain a small amount of a dynamic magnetic field. The dynamic magnetic field can be from 3% to 5% of the constant amplitude electromagnetic field.

The constant amplitude electromagnetic field system can also have a power line filter connected to a power input for supplying electric current to the magnetic modules. In addition, a surge arrestor can be selectively located at the power input to ensure even transmission of the electrical current to the wound magnetic wire coils.

In an alternative, non-limiting embodiment, the system can include a fail safe circuitry. The fail safe circuitry can be adapted to prevent power from connecting to the wound magnetic wire coil when the temperature around the wound magnetic wire coil exceeds a preset limit.

The system can be best understood with reference to the Figures. Referring now to the Figures.

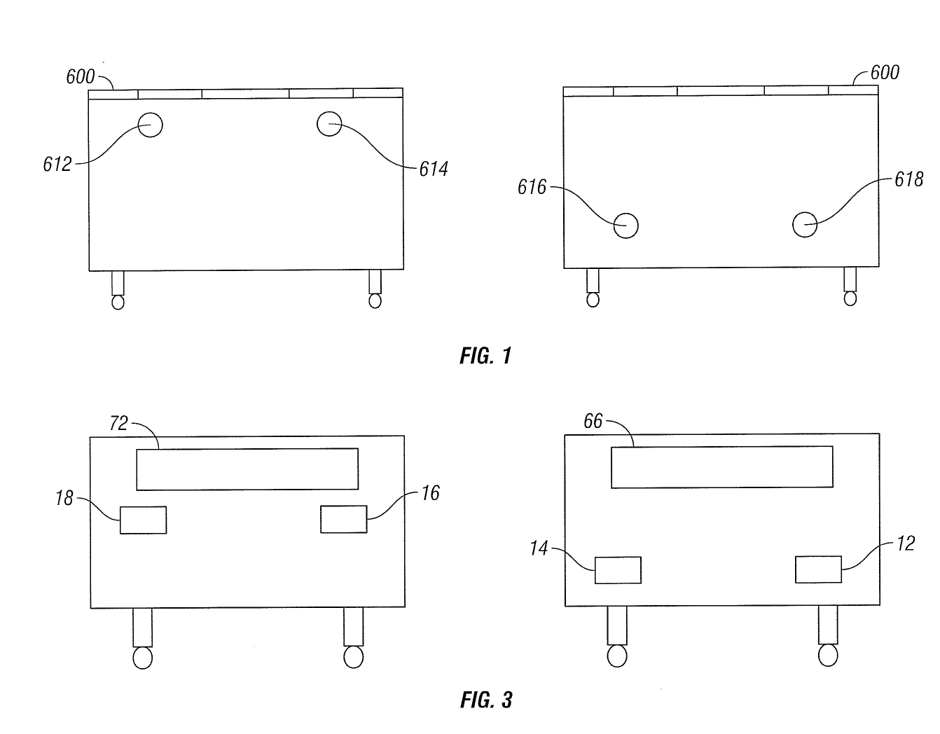

FIG. 1 is a prospective view of a non-limiting embodiment of the system. The left and right view of the housing 600 is depicted without the magnetic modules 12, 14, 16, and 18. The housing 600 has at least two holes 612 and 614 selectively disposed within the left side of housing 600. The housing 600 further has at least two more holes 616 and 618 selectively disposed within the right side of the housing 600.

The housing 600 can have a plurality of holes located at selective different positions. For example the housing can contain 4 holes with each hole having a different diameter for accommodating magnetic modules with varying sizes of apertures for receiving varying sizes of ends of limbs of the animal.

In addition, the holes can be located on the housing 600 in different alignments. For example, two modules can be aligned vertically perpendicular to each other so that the animal or person can insert a foot in one hole and a hand in another hole.

Another possible arrangement of the holes on the housing 600 can include two holes selectively placed so that the constant amplitude electromagnetic system can be positioned in a corner of a room, while still allowing access to the holes.

Another possible arrangement of the holes on the housing 600 can include two holes located on the left side of the housing 600, and two holes located on the right side of the housing 600. The holes on the left side of housing 600 can each be selectively located and sized to accommodate magnetic modules with apertures sized to receive a hand of a human. The holes on the left side of the housing 600 can each be selectively aligned and sized to accommodate magnetic modules with apertures sized to receive the feet of a human.

It is clear that there are several other possible arrangements of the holes and sizing of the holes located on the housing 600. The arrangements of the holes on the housing will be dictated by what is required by the specific use of the constant amplitude electromagnetic system.

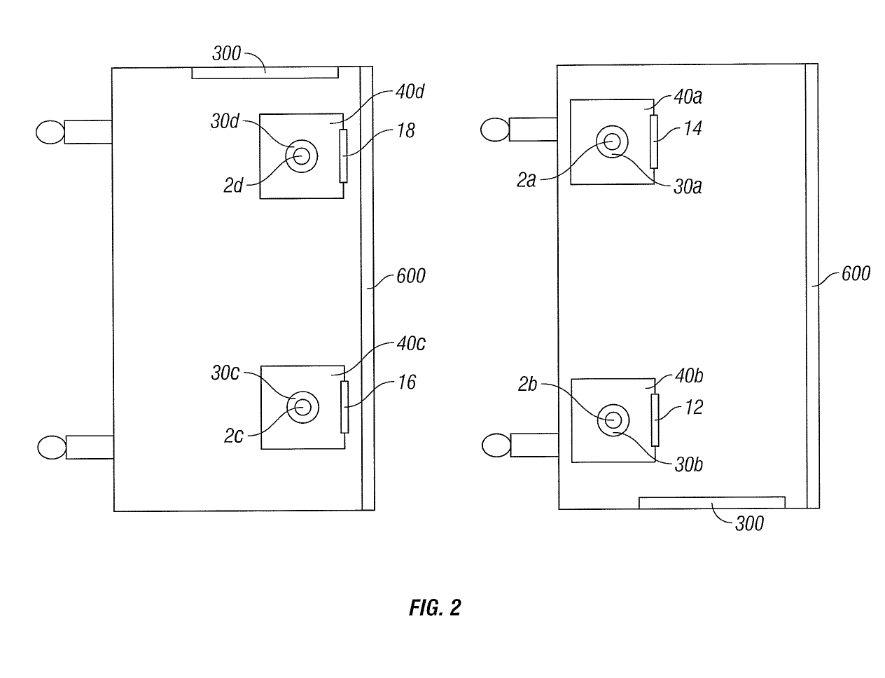

Referring now to FIG. 2, the holes 612, 614, 616, and 618 are operatively and selectively disposed upon the housing 600 to align with the magnetic modules 12, 14, 16, 18.

Referring now to FIG. 3, a first cooling reservoir 66 and a pressurizable cooling reservoir 72 are selectively disposed within housing 600. The first cooling reservoir 66 is selectively aligned to the left of the pressurizable cooling reservoir 72. The first cooling reservoir 66 and the pressurizable cooling reservoir 72 are operatively positioned at a similar elevation within the housing 600. In the embodiment depicted in FIG. 3 the pressurizable reservoir 72 and first cooling reservoir 66 are in an elevated position relative to the magnetic modules 12, 14, 16, and 18. The selective positioning of first cooling module 66 and the pressurizable reservoir 72 allows gravity to force cooling fluid into each of the magnetic modules when the system is off. This gravity feed ensures that each of the magnetic modules 12, 14, 16, 18 are supplied with cooling fluid.

Returning to FIG. 2, the magnetic modules 12, 14, 16, and 18 have apertures disposed within them. The aperture 2a is disposed within magnetic module 12, which is selectively, operatively aligned with hole 612, which is not depicted in FIG. 2 but is best shown in FIG. 1. The aperture 2b is selectively disposed within the magnetic module 14, which is selectively and operatively aligned with hole 610, which is not shown in FIG. 2 but is best depicted in FIG. 1. The aperture 2d is selectively disposed within the magnetic module 18, which is selectively, operatively aligned with hole 614, which is not shown in FIG. 2 but is best depicted in FIG. 1. The aperture 2c is selectively disposed within magnetic module 16, which is aligned with hole 616, which is not shown in FIG. 1 but is beat best depicted in FIG. 1.

The magnetic modules 12, 14, 16, and 18 include a ferromagnetic housing 40a, 40b, 40c, and 40d with non-ferromagnetic segments 29a, 29b, 29c, and 29d, which are not show in FIG. 2 but are depicted in FIG. 4, on a side forming apertures 2a, 2b, 2c, and 2d respectively.

Tubular sleeves 30a, 30b, 30c, and 30d are selectively disposed within the apertures 2a, 2b, 2c, and 2d respectively. The tubular sleeve 30a, which is substantially similar to tubular sleeves 30b, 30c, and 30d, is best depicted in FIG. 4. The tubular sleeves 30a, 30b, 30c, and 30d can be made from an insulating material, such as PVC.

The system should always have at least one aperture 2a, 2b, 2c, or 2d adapted to receive an end of a limb of an animal. In a preferred, non-limiting embodiment, the system includes four apertures 2a, 2b, 2c, and 2d adapted to receive a limb of an animal.

Referring now to FIG. 4, magnetic module 12 is depicted, however, it should be understood that each magnetic module 12, 14, 16, and 18 have the same components. Magnetic module 12 has a wound magnetic wire coil 38, which surrounds the aperture 2a.

The wound magnetic wire coil 38 is contained within a ferromagnetic housing 40, which has a non-ferromagnetic segment 29a. The wound magnetic wire coil 38 is operatively positioned upon a perforated spindle 10. The wound magnetic wire coil 38 generates an electromagnetic field. A separate wound magnetic wire coil 38 is located within each magnetic module 12, 14, 16, and 18. Each wound magnetic wire coil located within the magnetic modules 12, 14, 16, and 18 creates an independent electromagnetic field.

FIG. 4 also depicts the end cap 104 which is selectively attached to each tubular sleeve 30a, 30b, 30c, and 30d.

The perforated spindle 10 allows cooling fluid to flow through the wound magnetic wire coil 38 providing continuous cooling.

To help ensure adequate fluid flow to cool the tubular sleeve, spacers 78 and 79 are used to separate the perforated spindle 10 from the non-ferromagnetic segment 29a.

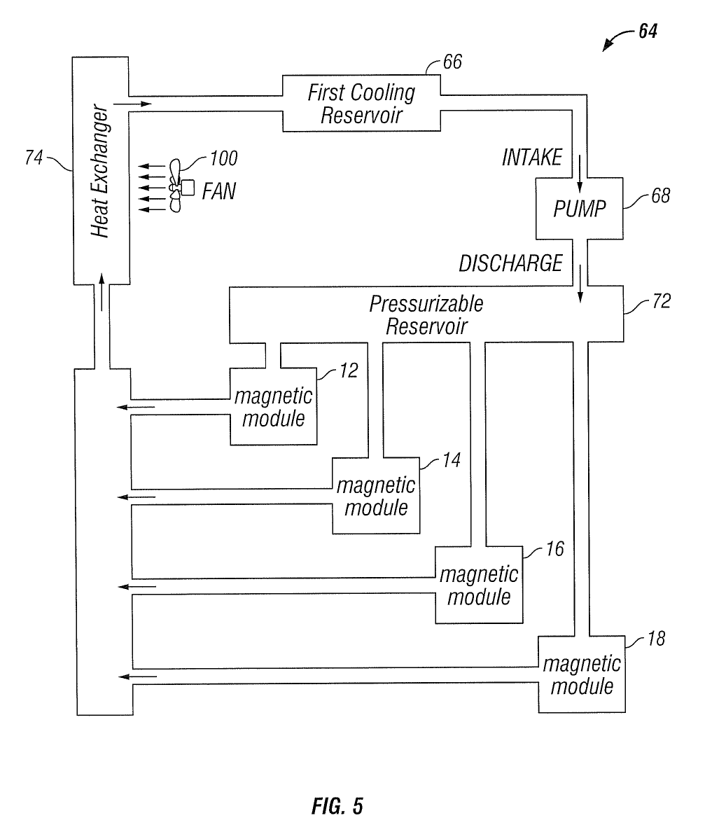

Referring now to FIG. 5, a schematic of the continuous flow cooling system 64 is depicted. The fluid can flow under the force of gravity or other means from the first cooling reservoir 66 to the pressurizable cooling reservoir 72. The fluid is selectively allowed to reach a homogenous equilibrium temperature within the pressurizable cooling reservoir 72. The pump 68 forces the fluid out of the pressurizable cooling tank into each module 12, 14, 16, and 18. When the constant amplitude electromagnetic system is not coupled to a power source, gravity can cause fluid to flow into the modules 12, 14, 16, and 18. This ensures that cooling fluid is always in the magnetic modules 12, 14, 16, and 18.

The cooling fluid that flows into each magnetic module 12, 14, 16, and 18 is at a substantially similar temperature. For example, but without limitation, if magnetic module 12 was not supplying a constant amplitude electromagnetic field and magnetic module 16 was supplying a constant amplitude electromagnetic field, an animal having an end of a limb in the magnetic module 12 would not know by the temperature of the magnetic module 12 that magnetic module 12 was not generating a constant amplitude electromagnetic field. The cooling fluid maintains the temperature throughout the magnetic modules 12, 14, 16, and 18 at less than 105 degrees Fahrenheit.

The pump 68 pressurizes the cooling fluid in the pressurizable cooling reservoir 72. The pump 68 does not generate more than 20 psi. It is contemplated that the pump 68 can be a switched magnetic coupling fluid circulation pump; however, other pumps that generate small pressures may be used.

FIG. 5 further depicts a heat exchanger 74. The heat exchanger 74 is used to continuously or periodically dissipate heat to the outside environment. It is contemplated that the heat exchanger can be coupled to a heat sink, allowing for both convection and conduction heat transfer.

In another alternative embodiment, the heat exchanger 74 and the pump 68 can be selectively located in a location remote from the housing 600.

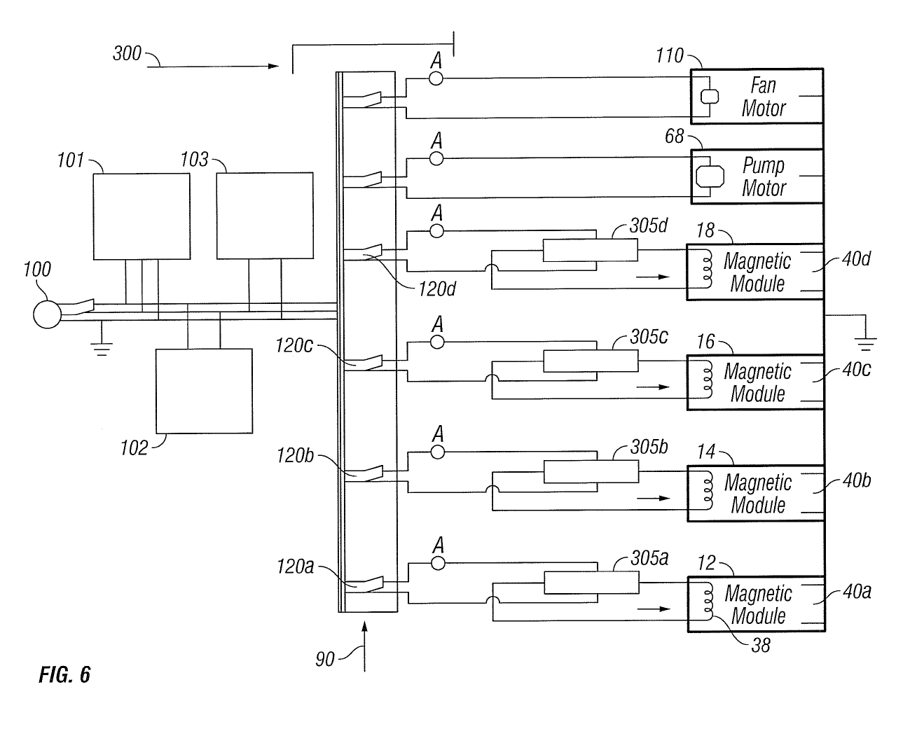

FIG. 6 is s schematic of an embodiment of the electrical system of the constant amplitude electromagnetic system. A plurality of switches 90 are selectively contained within a control panel 300, which is selectively located upon the housing 600. The plurality of switches 90 are used to couple power to each magnetic module 12, 14, 16, and 18.

Each switch, 120a, 120b, 120c, and 120d within the plurality of switches 90 are independently operated to couple direct current 62 to the separate magnetic modules 12, 14, 16, and 18 respectively.

Other switches can be selectively included within the plurality of switches 90 for controlling other parts of the constant amplitude electromagnetic system, such as the switchable continuous flow cooling system 64.

By the selective operation of the switches 120a, 120b, 120c, and 120d, the magnetic modules 12, 14, 16, and 18 can be operated independent of each other. When the AC power is coupled to the rectifiers 305a, 305b, 305c, 305d by the use of the switches 120a, 120b, 120c, and 120d the AC current is transformed into direct current 62, which is supplied to the wound magnetic coils 38. The direct current 62 can be independently supplied to the wound magnetic wire coils 38 in any of the magnetic modules 12, 14, 16, or 18 by selectively activating one of the switches 102a, 102b, 102c, or 102d respectively. When the direct current 62 is supplied to the wound magnetic coils 38 a constant amplitude electromagnetic field is generated.

The control panel 300, which contains the plurality of switches 90 is selectively located upon the housing 600. Housing 600 is not depicted in FIG. 6.

FIG. 6 further depicts an AC current power input 100. A surge arrestor 101 is located between the AC current power input 100 and the rectifiers 305a, 305b, 305c, 305d. The rectifiers 305a, 305b, 305c, and 305d independently transform the AC current into DC current 62 before the power is supplied to the magnetic modules 12, 14, 16, and 18. Additionally, a power line filter 102 is also disposed between the surge arrestor 101 and the rectifiers 305a, 305b, 305c, 305d. A fail safe circuit 103 is also shown disposed between the power line filter 102 and the rectifiers 305a, 305b, 305c, 305d.

For example, if magnetic module 12 was turned on, AC current would flow from the power input 100 through the surge arrestor 101, past the fail safe circuit 103, through the rectifier 305a, and then to the magnetic module 12. If the fan 110 or pump 68 were turned on simultaneously or independently from the magnetic module 12, the AC current would flow from the power input 100, through the surge arrestor 101, past the fail safe circuit 103, then to the fan 110 or pump 68.

The electromagnetic field is independently generated in each of the modules 12, 14, 16, and 18, which are best depicted in FIG. 2. The electromagnetic field has a closed form and is more spatially confined because of the attraction to the ferromagnetic housing 40a, 40b, 40c, 40d, which are best depicted in FIG. 2. The benefits include preventing interference with external devices, concentrating the field in a desired location, preventing unwanted exposure to other body parts, and providing lower levels of amplitude fields away from the housing.

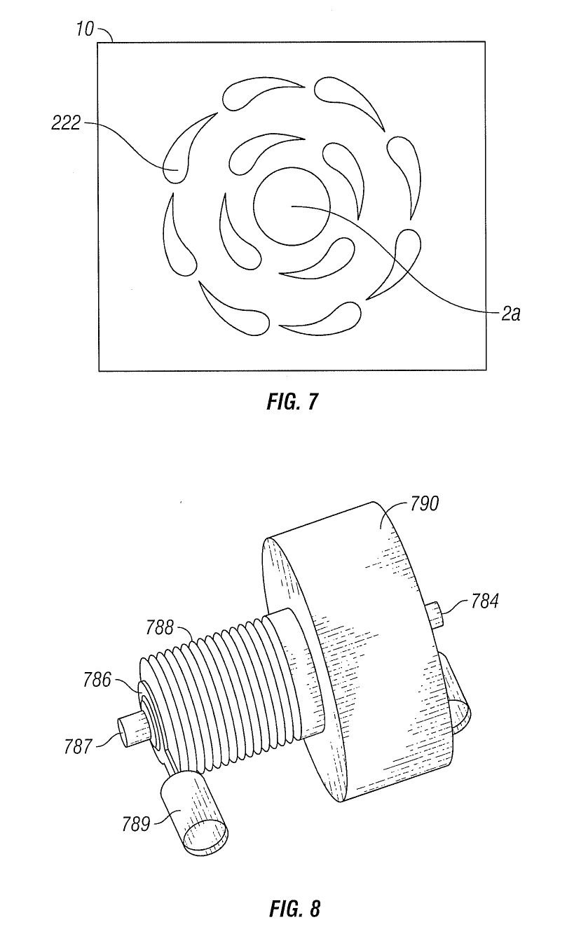

FIG. 7 is a view of the perforated spindle 10. The perforated spindle 10 has a plurality of elliptical openings 222. The elliptical openings 222 allow the cooling fluid to surround the wound magnetic wire coil 38 more efficiently. The perforated spindle 10 is shown having the aperture 2a, however it should be noted that the perforated spindle 10 is the same in each magnetic module.

The elliptical openings 222 are arranged so that the cooling fluid will flow towards the center of the wound magnetic wire coil 38, not shown in FIG. 7, producing a circular clockwise flow pattern before exiting. As the cooling fluid is exiting the wound magnetic wire coil 38 through the end of the perforated spindle opposite the end of cooling fluid entry, the elliptical openings 222 will cause the fluid to continue to flow towards the center of the wound magnetic wire coil 38.

FIG. 8 illustrates an exemplary electrical connector 780 for the housing 600 that reduces fluid leakage from the modules 12, 14, 16, and 18 via the electrical connection. Electrical connector 780 comprises a conductor post 784 that provides an external terminal connection 786 and an internal terminal connection 787 that span the modules 12, 14, 16, and 18 and connect the wound magnetic coil 38 to the wire 789 for transporting the direct current 62. The connector 780 can be attached to the housing 600 by threaded member 788. The connector can be attached using various washer and gasket configurations, which include a gasket or washer positioned between the external connector body 790 and the housing 600.

The above described embodiments of the electromagnetic system have been used in experimental treatment of certain diseases. In addition, there are several planed test to help better develop the system and to measure the effectiveness of the constant amplitude electromagnetic system on other diseases.

A non-limiting example of a method for performing a randomized double-blind medical clinical trial on a statistically significant number of participants using a constant amplitude electromagnetic system follows. The medical clinical trial could be designed to assess clinical symptoms response and blood properties response, after the constant amplitude electromagnetic system treatment, in participants who have complications due to a medical ailment, and to determine the safety of the constant amplitude electromagnetic system as a treatment modality.

The medical protocol should be determined before the medical clinical trial begins. The medical protocol for conducting the medical clinical trial could require that the medical clinical trial be conducted in a location where a population exists that are inflicted with the disease at a rate higher than that found in some areas of the United States. Further, a principal investigator (PI) could be required to be located in such a location, and that the PI be experienced with extensive treatment of the disease.

Additionally, another non-limiting example of the medical protocol of the medical clinical trial could be the requirement of measuring subjective severity of symptoms, and performing a blood test to show possible cell separation.

A non-limiting example of a medical protocol could be identifying primary objectives of the medical clinical trial. The primary objectives defined by the medical protocol could include: (1) determination of the extent of immediate pain relief as a result of treatment, (2) determination of the progression of pain relief from one interval in the treatment process to the next interval, (3) determination of the changes in blood properties as a result of treatment, (4) determination of the progression of changes in the blood properties from one interval in the treatment process to the next interval, (5) determination of the persistent effect of pain relief two weeks after treatment, (6) determination of the persistent changes in the blood properties two weeks after treatment, (7) determination of the extent of any visual acuity changes immediately after treatment, (8) determination of the progression of any visual acuity changes form one interval in the treatment process to the next interval, (9) determination of the extent of any persistent changes in visual acuity two weeks after treatment, (10) determination of the extent of immediate relief from numbness or tingling from as a result of treatment, (11) determination of the progression of relief from numbness or tingling from one interval in the treatment process to the next interval, and (12) determination of the persistent effect of relief from numbness or tingling two weeks after treatment. The medical protocol could also define secondary objectives of the clinical medical trial.

Another non-limiting example of the medical protocol could be set out parameters needed to conduct a Phase II or a Phase III trial to evaluate safety and to assess the treatment effect on clinical symptoms and blood properties of participants with complications due to medical ailment.

In another non-limiting example, the medical protocol could define a statistically significant number of participants to be one hundred participants, depending on the current standards of the medical industry. A statistically significant number can be a larger number of participants or a smaller number of participants depending on standards in the medical profession or determined standards for a specific medical clinical trial.

Yet another example of the medical protocol could be to define what a successful test result is, such as a net improvement effect for participants receiving actual treatment, less the net improvement effect for participants receiving the placebo treatment.

The machine operator will know the treatment control status, but subjects and other personnel will not. The system has a similar noise level and temperature whether in actual or placebo treatment mode, and controls for mode selection will not be visible to a participant.

After the medical protocol is defined and a statistically significant number of participants, which meet the participant criteria, are obtained, each of the participants will be assigned a participant number. A technician with no knowledge of the participants, except for their participation number, will operate a random number generator to classify which of the statistically significant number of participants will receive actual treatment, and which of the significant number of participants will receive placebo treatment.

The significant number of participants will be treated with the constant amplitude electromagnetic system based on the random number generator classification. The constant amplitude electromagnetic system is described in detail above and in the accompanying figures. The statistically significant number of participants will not know if they are receiving placebo treatment or actual treatment.

The constant amplitude electromagnetic system makes this possible because there will be no differences in temperature or noise between a magnetic module coupled to direct current and a magnetic module not coupled to direct current. The participants will experience the same temperature and noise level if they are receiving the actual treatment or the placebo treatment. This is due to the unique design of the constant amplitude electromagnetic system.

After the statistically significant number of participants are given treatment corresponding to the random number generator, classification data will be collected. The data will be collected using a technician without knowledge of which of the statistically significant number of participants were given actual treatment or placebo treatment.

The medical protocol for the medical clinical trial could require that the data that can be collected from the statistically significant number of participants, whether they received actually treatment or placebo treatment, can be the flowing: (1) the reduction in pain associated with the medical ailment, (2) improvement in eye sight measured by an eye exam, (3) the change in blood cells determined by results of a blood exam done before and after they received actual or placebo treatment.

The data will then be stored, analyzed, and correlated to the participant numbers. Then it will be determined whether, according to the medical protocol, the constant amplitude electromagnetic system is effective in treating the medical ailment.

The medical protocol can further require that the participants return two weeks after receiving the placebo treatment or the actual treatment and have additional data collected. At the follow-up visit two weeks after treatment using the constant amplitude electromagnetic system, the participant could be asked to submit an opinion as to whether he or she received an active or placebo treatment.

The medical protocol could further provide that after the data is collected during the follow-up visit, that the subject participant be informed as to whether he or she received actual treatment or placebo testament. For those actual treated participants their participation in the study will terminate at that time.

The medical protocol could allow for those participants receiving placebo treatment to be offered, and encouraged, to participate further with an open label treatment with the constant amplitude electromagnetic system. If the participant agrees to further treatment, the information measured and blood sampling described above will be followed. Their participation in the study will then be concluded.

The medical protocol could further require that each participant be given a form after the actual treatment or placebo treatment, for them to list any antibiotics or blood transfusions that they may have received during the two week interval between treatment with the constant amplitude electromagnetic system and the follow-up visit. The PI will use that information as pestilential confounding factors in his assessment of the efficacy of the constant amplitude electromagnetic system treatment.

The medical protocol could define the study population as male or female participants, ages 18-80, who meet eligibility requirements. Participants from all races or ethnic groups could be invited to participate.

The medical protocol could have the following conditions for a participant to be enrolled in the study. First, participants could be required be 18-80 years old. Second, medical records adequate to document that participants have the selected medical ailment could b required. Finally, written informed consent conforming with to IRB guidelines must be given.

The medical protocol could define any of the following conditions as eliminating a participant from participating in this clinical medical trial. (1) Inadequate records to establish proof of having been diagnosed with the medical ailment, (2) pregnancy or breast feeding, (3) receipt of blood transfusions within thirty days prior to clinical medical trial, (4) receipt of antibiotic treatment within 14 days prior to the medical clinical trial, (5) contraindication to MRI, (6) prior experience with the constant amplitude electromagnetic system, (7) unwillingness or inability to conform to all the steps required to complete the study.

The constant amplitude electromagnetic system, incorporated with the method for performing a randomized medical clinical trial, allows for results that possess FDA approval and medical community acceptance. The prior tests on the effects of electromagnetic treatment have been ineffective because of the delay in recordable results, the inability to provide placebo treatment to one participant while simultaneously providing actual treatment to another participant, and inconsistent exposure to the electromagnetic field.

The described electromagnetic system allows for results to be measurable in a relatively short period, due to the concentrations and intensity of the constant amplitude electromagnetic field.

In addition, the constant electromagnetic field is capable of delivering a constant intensity of a constant amplitude electromagnetic field regardless of the operator's experience or the timing or location of the actual treatment.

Finally, the constant amplitude electromagnetic system is capable of providing actual treatment to one participant and placebo treatment to another participant simultaneously. The switchable continuous flow cooling system prevents either participant from knowing if they are receiving placebo treatment or actual treatment. This is possible because the switchable continuous flow cooling system maintains a similar temperature within each magnetic module, even when one magnetic module is coupled to direct electric current and the other magnetic modules are not coupled to direct electric current.

There are several known and contemplated uses for the constant amplitude electromagnetic system.

It is contemplated that an embodiment of the constant amplitude electromagnetic system can be used under medical supervision for adjunctive therapy for the treatment of medical diseases and conditions. The constant amplitude electromagnetic system of the invention may be indicated for use in stimulating neuromuscular tissues for bulk muscle excitation in the legs or aims for rehabilitative purposes, for treating arthritis, for treating diabetes, for treating HIV, for treating AIDS, for treating shingles, for treating soft tissue damage, for treating sickle cell anemia, for treating cardiopulmonary disease, for treating cancer, for treating chronic pain, or for treating a similar disease.

Indication for use of muscle stimulators include, but are not limited to relaxation of muscle spasms, increasing blood circulation, muscle reduction, prevention or retardation of disuse atrophy, immediate post-surgical stimulation of calf muscles to prevent venous thrombosis, or maintaining or increasing range of motion.

Magnetic therapy systems have been used for the relief of pain with demonstrated efficacy and safety. Other similar systems have delivered electromagnetic energy either transcutaneously or invasively for pain relief and to promote healing and bone growth.

Electrical and magnetic nerve stimulation has been used for over one hundred years, but more importantly over the past 20 years, with sound medical studies and supervision. The use has shown well-established positive results in pain relief without negative side effects.

Each stimulation system may have different body responses based on its particular delivery of electromagnetic energy. There exists almost unlimited possible variations in the electromagnetic field through changes in current, voltage, frequency, waveform, and length of delivery of the energy.

The constant amplitude electromagnetic system of the present invention delivers electromagnetic energy at very low levels, well within parameters of other FDA approved magnetic stimulator systems. Its purpose and intended use is similar to other stimulator systems: to relieve pain and to possibly affect the electric potential of an unhealthy human blood cell. These systems are intended to be used for the relief of pain which has been determined by adequate medical diagnosis as being chronic or in response to conditions known and being treated.

The body cell functions, being a complicated combination of electrical and chemical ion interactions, may be directly affected by the constant amplitude electromagnetic field.

Alternative embodiments of the system described herein provide delivery of electromagnetic energy in a form which provides many of the same type of cell interactions as other magnetic stimulation systems. The importance of this system is that it provides pain relief, but potentially affects greater body volume and in some cases a longer lasting treatment effect, at significantly safer electromagnetic levels, with an inherently safer delivery system and a more easily performed procedure.

The electromagnetic system has been tested on people with diabetes. Diabetes is the most common chronic disease in the developed countries, with more than one hundred million cases diagnosed worldwide, and sixteen million diagnosed in the United States. It is the third most common cause of death in the U.S. Approximately one percent of all diabetes patients undergo amputations, a rate fifteen times higher than the non-diabetic population.

An estimated seventy percent of diabetic patients have nerve damage that impairs feeling in their feet. Fifteen percent of all diabetics eventually will develop a foot ulcer. Among those with ulcers, one in four will lose a foot. Every year, over 86,000 amputations are performed as a result of diabetes, and studies show that about one half of those who have a foot or leg amputated will lose the other one within five years.

Blindness and visual disability are caused due to diabetes mellitus. The blood vessels in the retina are damaged due to this chronic autoimmune disease which eventually results in loss of vision. It is possible to suggest from findings, consistent from study to study, that approximately two percent of people become blind after fifteen years of diabetes, while severe visual handicaps are developed by about ten percent of diabetics. Loss of vision from certain types of glaucoma and cataracts may be more common in people with diabetes than in those without it.

The constant amplitude electromagnetic treatment system has been tested, over the span of several years, on thousands of human participants. There has been an on-going exchange of information regarding the constant amplitude electromagnetic system with the United States Food and Drug Administration (FDA). The FDA has ruled that it is a “non-significant-risk system.” No adverse side effects have been noted in the participants treated, and the number of experimental treatments administered exceeds fifteen thousand. The constant amplitude electromagnetic treatment system has been observed to be effective to a significant degree in reducing pain in a wide variety of diseases, injuries, and other medical conditions. Participants with various diabetic complications are among those whose pain scores have been shown reduced after treatments.

Type 1 diabetes requires that insulin be replaced directly. Type 2 diabetics have various drugs available to enhance the production of natural insulin rather than replacing it. Various combinations of drugs may be tried as the disease progresses. Eventually, natural insulin may completely fail and insulin replacement is needed.

Tight control of blood glucose levels delays the onset and slows the progression of neuropathy. Certain drugs and even natural supplements have proved helpful. Tight insulin control is needed to help prevent retinopathy. Once damage to the eye develops, surgery may be needed.

Experimental treatment with the constant amplitude electromagnetic treatment system has been demonstrated in previous studies to be an effective treatment modality for reducing the severity of symptoms of both diabetic neuropathy and diabetic retinopathy.

Sickle cell disease is an inherited blood disorder characterized by defective hemoglobin. It affects millions of people throughout the world and approximately 72,000 people in the U.S. It is present in one of every 500 African-American births.

Normal hemoglobin cells are smooth and round, allowing for ease in moving through blood vessels. Sickle cell hemoglobin molecules are stiff and form into the shape of sickle or a scythe. They tend to cluster together, and cannot easily move through blood vessels. The cluster causes a blockage and stops the movement of oxygen-carrying blood. Sickle cells die after about 10 to 20 days, unlike normal hemoglobin cells, which live for up to 120 days. This results in a chronic short supply of red blood cells, which causes anemia. In this condition most or all of the normal hemoglobin (HbA) has been replaced with the sickle cell hemoglobin (HbS). This is referred to as HbSS. It is the most common and most severe form of the sickle cell variations. These persons suffer from a variety of complications due to the shape and thickness of the sickle cell. Severe and chronic anemia is also a common characteristic for children with HbSS. (www.umm.edu).

Current specific treatment regimens for those who suffer sickle cell anemia are determined by the treating physician according to the participant's age, overall health, and medical history. Other factors include the extent of the disease; tolerance for specific medications, procedures, or therapies; expectations for the course of the disease; and the patient's opinion or preference. Treatments may include pain medications (for sickle cell crises); drinking plenty of water daily (eight to ten glasses) or receiving fluid intravenously (to prevent and treat pain crises); blood transfusions to relieve anemia and to prevent stroke, and to dilute the HbS with normal hemoglobin to treat chronic pain; acute chest syndrome, splenic sequestration, and other emergencies; folic acid (to help prevent severe anemia); hydroxyurea, a medication that helps reduce the frequency of pain crises and acute chest syndrome, and may decrease the need for frequent blood transfusions; bone marrow transplants, which has been successful in curing some persons with sickle cell disease, and/or penicillin (to prevent infections).

The established efficacy of the constant amplitude electromagnetic system to either replace or augment any of the established treatment modalities would be a highly desirable event. Anecdotal evidence suggests a strong possibility of this event being accomplished.

Another contemplated use for the constant amplitude electromagnetic system is in treating people affected with Acquired Immune Deficiency syndrome (AIDS). AIDS is an impairment of the body's ability to fight disease. It leaves the affected individual vulnerable to illnesses that a healthy immune system might overcome. AIDS patients are susceptible to diseases called opportunistic infections. These are illnesses due to organisms commonly found in the environment, and harmful only to an individual with a weakened immune system. This lack of an immune system alters in important ways, the lives of these patients. AIDS affects approximately two million people in the United States.

AIDS patients are affected by early symptoms that are usually benign and inconspicuous but may include fatigue; loss of appetite; fever; night sweats; swollen glands (enlarged lymph nodes) in the neck, armpits, or groin; unexplained weight loss, diarrhea, persistent cough, and various skin lesions. However, the symptoms may persist for months or worsen as opportunistic diseases exploit the body's collapsed defenses. Many (about 52%) develop an unusual pneumonia caused by the protozoan Pneumucysti carinii. Another third of patients exhibit a rare cancer of the skin, Kaposi's sarcoma (KS) or contract one of the many opportunistic diseases caused by fungi (yeasts), viruses, bacteria, and protozoans.

AIDS patients may be divided into three groups: (i) AIDS patients having only Kaposi's sarcoma (KS) and no other symptoms, (ii) AIDS patients having only severe opportunistic infections, such as Pneumucysti carinii pneumonia (PCP), or Candida (thrush), (iii) AIDS patients having both KS and opportunistic infections. The third group suffers the most severe changes in their immune systems, while patients with KS alone are the least severe.

The present treatment of AIDS includes the use of various pharmaceuticals; AL-721, Ampligen, Azidothymidine (AZT), Recombinant soluble CD4 (T4), Dextran sulfate, Dideoxycytidine (ddC), Foscarnet, Interferons, Ribaviron, among others, which are used alone or in combination. One goal of AIDS treatment is the complete suppression of the crisis without the development of collateral effects related to the medicine. Medicinal therapies are not sufficient to control the AIDS crisis. The recurring crisis has a long lasting effect upon the cognitive and psychosocial development, as well as on the quality of life.

Recently, magnetic stimulation has been used with success for the treatment of AIDS. Although the mechanism of action remains unknown, the following hypotheses have been postulated: The first is the cancellation of AIDS symptoms is related to the destructive interference of the coherent magnetic wave, produced by the stimulation. The second is that the magnetic or electrical properties of the affected cells are changed. A third hypothesis is that the magnetic stimulation produces an inhabition of the viral activity in the region that produces dysfunction of the immune system. A fourth hypothesis is that the magnetic fields alter the properties at the biologic membranes regulating a variety of ionic pumps.

Another hypothesis is based on studies that have shown that T4 cells directly or indirectly regulate immune function. Magnetic stimulation suppressed the activity of the endogenous opioid peptides. Some drugs, such as phenotoin, Phenobarbital, and sodium valproate reduce the concentration of beta-endorphins in rats, suggesting that the mechanism of action of these drugs may be partly behind the interaction with the endogenous opioid peptides.

In still further study, the application of magnetic fields has been shown to influence the activity of purkinje cells in the brain, and because it is known these cells participate heavily in the initiation, as well as the propagation of the cerebral activity, it is possible that the effects of magnetic fields can affect cerebral activity. The reduction of AIDS symptoms may be related in part to the disease's interference with cerebral functions. Autopsies reveal signs of neurological disease in 80 percent to 90 percent of people who die from AIDS.

In yet another study, the pineal gland is believed to be a magnetism-sensitive organ. It is conceivable that the effects of magnetic fields upon AIDS may be mediated via pineal gland. It has been demonstrated that magnetic fields affect the pineal glands producing structural changes, reduction of serotonin-N-acetyl tranferase and hydroxyindol-orthomethyl transferase; reduction in the production of melatonin; behavior opposite that which the rat exhibits with the administration of melatonin. All of these discoveries indicate that changes in the magnetic environment can influence the production of melatonin and the modulation of the circadian rhythms dependent on the pineal gland.

The following evidence supports the association between the pineal gland activity and AIDS: (a) melatonin influences the electric activity of the brain in human beings and animals, (b) administration of tritiated melatonin shows an accumulation in the cerebral structures which integrate cerebral activity (i.e., the hippocampus and the cerebellum); (c) the administration of melatonin produces dischronation of the electroencephalogram; (d) the intraperitoneal administration of melatonin in small doses augments the discharge of neurons from the red nucleus; and (e) a pinealectomy produces general paroxysmal crisis of slow waves with high amplitudes of cephalic center organ.

The following examples are included to demonstrate alternative, non-limiting embodiments of the invention. It should be appreciated by those skilled in the art that the techniques disclosed in the examples which follow represent alternative non-limiting techniques discovered by the inventor to function well in the practice of the invention. However, those skilled in the art should, in light of the present disclosure, appreciate that many changes can be made in the specific embodiments which are disclosed and still obtain a like or similar result without departing from the spirit and scope of the invention.

One example of a use of the constant amplitude electromagnetic system involves a female participant who was suffering from sickle-cell anemia. The participant received three treatments using the constant amplitude electromagnetic system as described herein. Blood was drawn before and after the third treatment. Peripheral smears were evaluated for differential RBC counts.

Pre-treatment sickle-cell percentage was 25% (75% non-sickle cells). Post treatment sickle-cell percentage was 10% (90% non-sickle cells). It is contemplated to expand these studies to greater number of patients to further support these results.

In another example, one participant had epileptic attacks for 28 years, with an occurrence from 1 to 2 times per month, and was selected for treatment by the constant amplitude electromagnetic system described herein. After having been treated by the constant amplitude electromagnetic system for about two months, she substantially improved because at least two thirds of her attacks were relieved.

In a third example, an epileptic woman who had experienced at least 8 attacks per month for 34 years was also exposed to the constant amplitude electromagnetic field, and at the end of a six-week period, she was evaluated. She had only four attacks, which occurred during the first four weeks of the treatment period, while during the last three weeks of the treatment no attacks occurred.

In a fourth example, an epileptic woman who had suffered attacks for the last 9 years, was selected for constant amplitude electromagnetic treatment for a period of approximately two months. Prior to her treatment, this participant had convulsive attacks with a frequency of 3 to 12 per month. Her attacks diminished to 7 times during the two month evaluation period, no attacks having occurred during the last ten days of the period. The treatment was suspended because she became pregnant.

In a fifth example, a 33 year old epileptic woman, who had from 1 to 3 attacks per month for 15 years, was treated for ten weeks. The participant had only one attack at the beginning of the treatment, and no attacks occurred for the remaining period of constant amplitude electromagnetic treatment.

In a sixth example, a 19 year-old woman, who had from 8 to 20 epileptic attacks per month for 4 years, was treated by the constant amplitude electromagnetic field system, and within the first 5 days this patient had 16 attacks. During the following 10 days, the frequency of the attacks diminished to 4 attacks in this latter period, and during the last 10 days of the evaluation she had only 3 attacks. A significant reduction in the occurrence of the epileptic attacks was noted.

The participants described in examples 2-6 have been treated to control their convulsive attacks by means of anticonvulsive medicine, such as Phenobarbital, phenytoin, valproic acid, primidone, carbamazepine, etc. Except for the third participant, all of the participants remain under treatment by the constant amplitude electromagnetic field system described herein, and encouraging results have been obtained for each participant.

In a seventh example, the effectiveness of pain relief using the compositions and methods of the present invention has been documented in participants undergoing treatment using the constant amplitude electromagnetic system and method of providing described herein. An open label study of 79 subjects was made. The secondary effect of pain relief was observed and documented in participants being treated. The results of a study documenting pain relief are presented in the Provisional Application incorporated by reference in its entirety. The effect of the constant amplitude electromagnetic system was measured for pain relief for a wide range of maladies causing pain.

Included in the study were subjects suffering from poor circulation, arthritis, fibromyalgia, multiple sclerosis, back pain, neuropathy, diabetes, and other diseases. For 1,195 painful locations compared before treatment and after treatment, there was an average reduction in pain level after treatment of 2.39 points, on a scale of 0 being no pain and 10 being unbearable pain.

Although the description of the preferred embodiment has been presented, it is contemplated that various changes could be made without deviating form the spirit of the present invention.

What is claimed is:

1. A method for performing a randomized double-blind medical clinical trial on a statistically significant number of participants using a constant amplitude electromagnetic system, wherein the constant amplitude electromagnetic system comprises:
a housing; at least one magnetic module contained within the housing, wherein each magnetic module comprises: a ferromagnetic housing; comprising a non-ferromagnetic segment forming an aperture; a wound magnetic wire coil on a perforated spindle having a coil diameter greater than a coil depth surrounding the aperture and located between the non-ferromagnetic segment and the ferromagnetic housing, wherein the wound magnetic wire coil generates a constant amplitude electromagnetic magnetic field within the aperture when coupled to a direct electrical current; and a tubular sleeve is contained within the aperture, and wherein each aperture is sized to receive at least one limb of a at least one of the at least two participants; and a switchable continuous flow cooling system contained within the housing for directing cooling fluid from a first cooling fluid reservoir to a pressurizable cooling fluid reservoir and pumping the pressurized cooling fluid into each ferromagnetic housing and around each wound magnetic wire coil to cool each wound magnetic wire coil maintaining each aperture at a substantially similar temperature relative to each other; and wherein the housing comprises at least one extrusion operatively aligned with each aperture of the at least one magnetic module;
forming a medical protocol for use with the constant amplitude electromagnetic system to determine if exposure to a constant amplitude electromagnetic field will affect at least one defined ailment;
selecting participant criteria for an actual treatment using the constant amplitude electromagnetic system;
assigning each of a statistically significant number of participants a participant number;
using a random number generator to classify which of the statistically significant number of participants receives the actual treatment or receives a placebo treatment;
treating each of the statistically significant number of participants based on the random number generator classification without advising any of the statistically significant number of participants whether any of the statistically significant number of participants are being treated with the constant amplitude electromagnetic field using the constant amplitude electromagnetic system;
analyzing data using a technician without knowledge of which of the statistically significant number of participants were given actual treatment or the placebo treatment; and
storing the analyzed data and correlating the stored data to the participant numbers.

2. The method of claim 1, wherein the defined ailments are arthritis, diabetes, HIV, AIDS, shingles, soft tissue damage, sickle cell anemia, cardiopulmonary disease, cancer, chronic pain, or a similar disease.

3. The method of claim 1, wherein the statistically significant number of participants are humans or animals.

4. The method of claim 1, wherein the constant amplitude electromagnetic field within each aperture is a substantially homogeneous constant amplitude electromagnetic field.

5. The method of claim 1, wherein the volume of each aperture is constant.

6. The method of claim 1, further comprising from 2 to 4 magnetic modules, wherein each magnetic module is adapted to independently and simultaneously perform the actual treatment.

7. The method of claim 6, further comprising independently and simultaneously providing actual treatment for at least one of the statistically significant number of participants, and independently providing the placebo treatment for at least one of the statistically significant number of participants.

8. The method of claim 1, wherein the form of treatment given to the statistically significant number of participants is hidden from the statistically significant number of participants.

9. The method of claim 1, wherein the constant amplitude electromagnetic system for treating the statistically significant number of participants complies with the European Union International Medical Device Directives (MDD) and International Standards Organization's standards 60601-1, 60601-1-1, and 60601-1-2, which are required in 2006.

10. The method of claim 6, wherein the switchable continuous flow cooling system cools each magnetic module simultaneously maintaining each of the magnetic modules at a substantially similar temperature relative to each of the other magnetic modules.

11. The method of claim 1, wherein the constant amplitude electromagnetic field system comprises: connecting a AC power supply to a plurality of rectifiers, and wherein the plurality of rectifiers independently create the direct electric current, and wherein each magnetic module is operatively independently connected to a rectifier from the plurality of rectifiers providing the direct electric current independently to each magnetic module, and wherein the constant amplitude electromagnetic system further comprises a power line filter connected between the plurality of rectifiers and the AC power supply.

12. The method of claim 11, wherein the constant amplitude electromagnetic system further comprises a surge arrestor connected between the power line filter and the AC power supply.

13. The method of claim 1, wherein the constant amplitude electromagnetic system further comprises a fail safe circuitry adapted to prevent power from connecting to the wound magnetic wire coil when temperatures around the wound magnetic wire coil exceed a preset limit.

14. The method of claim 1, wherein the participant criteria is a combination of gender, age, ethnicity, pregnancy, health level, or any other medically relevant condition.

15. The method of claim 1, wherein an operator of the constant amplitude electromagnetic system cannot modify the constant amplitude electromagnetic system.

16. A method for performing a randomized double-blind medical clinical trial on a statistically significant number of participants using a constant amplitude electromagnetic system comprising the steps of:
forming a medical protocol for use with the constant amplitude electromagnetic system to determine if exposure to a constant amplitude electromagnetic field will affect at least one defined ailment;
selecting participant criteria for an actual treatment using the medical protocol;
wherein the actual treatment comprises:
treating at least one of a statistically significant number of participants using a constant amplitude electromagnetic field; providing at least one constant amplitude electromagnetic field concentrated at a center location; continuously maintaining the area around the constant amplitude electromagnetic field at a temperature less than 105 degrees Fahrenheit; placing an end of a limb of at least one of the statistically significant number of participants at the center location; exposing the end of the limb of at least one of the statistically significant number of participants to the constant amplitude electromagnetic field for a length of time ranging from 10 minutes to 3 hours to simultaneously treat venous and arterial blood cells and nerve endings in the end of the limb of at least one of the statistically significant number of participants;
assigning each of the statistically significant number of participants a participant number;
using a random number generator to classify which of the statistically significant number of participants receives the actual treatment or receives a placebo treatment;
treating each of the statistically significant number of participants based on the random number generator classification without advising any of the statistically significant number of participants whether any of the statistically significant number of participants are being treated with the constant amplitude electromagnetic field using the constant amplitude electromagnetic system;
analyzing data using a technician without knowledge of which of the statistically significant number of participants were independently given actual treatment or the placebo treatment; and
storing the analyzed data and correlating the stored data to the participant numbers.

17. The method of claim 16, wherein the defined ailments are arthritis, diabetes, HIV, AIDS, shingles, soft tissue damage, sickle cell anemia, cardiopulmonary disease, cancer, chronic pain, or a similar disease.

18. The method of claim 16, wherein the statistically significant number of participants are humans or animals.

19. The method of claim 16, wherein the constant amplitude electromagnetic field within each center location is a substantially homogeneous constant amplitude electromagnetic field.

20. The method of claim 16, wherein the selective placement of the end of the limbs of the statistically significant number of participants by an operator at the center locating does not affect the actual treatment or the placebo treatment.

21. The method of claim 16; further comprising from 2 to 4 constant amplitude electromagnetic fields, wherein each constant amplitude electromagnetic field is adapted to independently and simultaneously perform the medical protocol.

22. The method of claim 21, further comprising simultaneously maintaining the temperature of each area around the constant amplitude electromagnetic fields at a substantially similar temperature relative to each other.

23. The method of claim 16, further comprising automatically stopping the constant amplitude electromagnetic field when the constant temperature at the area around the constant amplitude electromagnetic field exceeds a pre-set limit.

24. The method of claim 16, further comprising providing from 2 to 4 constant amplitude electromagnetic fields wherein each constant amplitude electromagnetic field is adapted to independently treat the ends of the limbs of the statistically significant number of participants simultaneously.

25. The method of claim 24, further comprising providing the actual treatment to at least one of the statistically significant number of participants, and independently providing the placebo treatment to at least one of the statistically significant number of participants simultaneously.

26. The method of claim 24, further comprising simultaneously maintaining the temperature of each area around the constant amplitude electromagnetic fields at a substantially similar temperature relative to each other.

27. The method of 16, wherein the participant criteria is a combination of gender, age, ethnicity, pregnancy, health level, or any other medically relevant condition.
